(19) United States
(12) Patent Application Publication
(10) Pub. No.: US 2019/0391157 A1
(43) Pub. Date: Dec. 26, 2019

(54) MASS SPECTROMETRY IMAGING OF BENIGN MELANOCYTIC NEVI AND MALIGNANT MELANOMAS

(71) Applicants: Rossitza Zinovieva Lazova, Los Gatos, CA (US); Erin Heather Seeley, Morgantown, WV (US); Katy Ryan Smoot, Morgantown, WV (US)

(72) Inventors: Rossitza Zinovieva Lazova, Los Gatos, CA (US); Erin Heather Seeley, Morgantown, WV (US); Katy Ryan Smoot, Morgantown, WV (US)

(73) Assignees: New River Labs, LLC, Morgantown, WV (US); Yale University, New Haven, CT (US)

(21) Appl. No.: 16/507,656

(22) Filed: Jul. 10, 2019

Related U.S. Application Data
(63) Continuation-in-part of application No. 15/288,419, filed on Oct. 7, 2016.
(60) Provisional application No. 62/238,313, filed on Oct. 7, 2015.

(51) Int. Cl. G01N 33/68; H01J 49/00; H01J 49/16; A61N 5/10; G01N 1/30

(52) U.S. Cl. CPC G01N 33/6851; H01J 49/0031; H01J 49/164; H01J 49/0036; G01N 2800/7028; G01N 1/30; G01N 33/6881; G01N 2001/302; A61N 5/10

(57) ABSTRACT

A method of differentiating benign melanocytic nevi from malignant melanomas is disclosed. The method generally includes treating and subjecting a skin lesion sample from a patient to mass spectrometry to obtain a mass spectrometry proteomic profile. This profile is compared to mass spectrometry proteomic profiles of reference samples, which include benign melanocytic nevi and/or malignant melanomas. Classification of the skin lesion sample as a benign melanocytic nevus or a malignant melanoma is based on similarities and/or difference between the mass spectrometry proteomic profiles.

### CROSS REFERENCE TO RELATED APPLICATIONS

This application is a continuation-in-part of U.S. application Ser. No. 15/288,419, filed Oct. 7, 2016, which claims priority to U.S. Provisional Application No. 62/238,313, filed Oct. 7, 2015, the entire content of each of which is hereby incorporated by reference.

### TECHNICAL FIELD

The present invention relates to mass spectrometry profiling, also known as mass spectrometry profiling, and in particular, systems and methods of using mass spectrometry profiling to differentiate between benign melanocytic nevi and malignant melanomas.

### BACKGROUND

Matrix-assisted laser desorption ionization (MALDI) mass spectrometry imaging (MSI) may be used to analyze metabolites, peptides and proteins, DNA segments, and lipids directly from tissue sections with spatial fidelity. MALDI MSI may be performed on either fresh frozen or formalin-fixed, paraffin-embedded (FFPE) tissue specimens. MSI may be used to elucidate molecular profiles of different tumor types and grades including brain, oral, lung, breast, gastric, pancreatic, renal, ovarian and prostate cancers. Conventional MSI may suffer from one or more of the following limitations: spatial resolution, mass accuracy, spectral resolution, sensitivity, robustness, reproducibility, requirements for sample preparation, and degree of technical difficulty. Accordingly, more efficient mass spectrometry imaging systems and methods of making and using the same are desirable.

### SUMMARY

The present invention relates to a method of differentiating benign melanocytic nevi from malignant melanoma. The method may generally comprise treating a sample from a patient; subjecting the sample to mass spectrometry to obtain a mass spectrometric proteomic profile of the sample; comparing the mass spectrometric proteomic profile to a known normal tissue proteomic profile, benign melanocytic nevus proteomic profile, and/or a known malignant melanoma proteomic profile; and classifying the sample as one of a benign melanocytic nevus or a malignant melanoma based on similarities and/or differences between the proteomic profiles. The method may comprise classifying the sample as one of at least three subtypes of benign melanocytic nevi. The method may comprise classifying the sample as one of at least seven subtypes of malignant melanoma and/or a metastatic melanoma.

### BRIEF DESCRIPTION OF THE DRAWINGS

The present invention described herein may be better understood by reference to accompanying figures, in which:

FIG. 1 shows a histology guided mass spectrometry profiling workflow according to the present invention.

FIG. 2 shows an imaging and statistical analysis according to the present invention.

FIG. 3 shows images of benign melanocytic nevus and malignant melanoma as defined according to the present invention.

FIG. 4 shows a proteomic analysis signature according to the present invention.

FIG. 5 shows a comparison of an average spectrum for benign melanocytic nevus (top) and malignant melanoma (bottom) obtained using mass spectrometry the present invention.

FIG. 6 shows a gel view of the average spectra shown in FIG. 5.

FIG. 7 shows a histology guided mass spectrometry profiling workflow according to the present invention.

FIG. 8 shows results for a method for differentiating benign melanocytic nevi from malignant melanomas according to the present invention.

FIG. 9 shows a histogram of HGMS proteomic profile scores of known benign (green) and malignant samples (red) for a method for differentiating benign melanocytic nevi from malignant melanomas according to the present invention.

### DETAILED DESCRIPTION

Throughout this description and in the appended claims, use of the singular includes the plural and plural encompasses the singular, unless specifically stated otherwise. For example, although reference is made herein to “a” profile, “a” protein, “a” section, and “an” image, one or more of any of these components and/or any other components described herein may be used.

As generally used herein, the terms “including” and “having” mean “comprising”. The word “comprising” and forms of the word “comprising”, as used in this description and in the claims, does not limit the present invention to exclude any variants or additions.

As generally used herein, the term “about” refers to an acceptable degree of error for the quantity measured, given the nature or precision of the measurements. Typical exemplary degrees of error may be within 20%, 10%, or 5% of a given value or range of values. Alternatively, and particularly in biological systems, the terms “about” refers to values within an order of magnitude, potentially within 5-fold or 2-fold of a given value.

All numerical quantities stated herein are approximate unless stated otherwise. Accordingly, the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as measured.

Any numerical range recited in this specification is intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this disclosure is intended to include all lower numerical limitations subsumed therein and any minimum numerical limitation recited in this disclosure is intended to include all higher numerical limitations subsumed therein. Accordingly, Applicants reserve the right to amend this specification, including the claims, to expressly recite any sub-range subsumed within the ranges expressly recited herein.

This disclosure describes various features, aspects, and advantages of various embodiments of the invention. It is understood, however, that this disclosure embraces numerous alternative embodiments that may be accomplished by combining any of the various features, aspects, and advantages of the various embodiments described herein in any combination or sub-combination that one of ordinary skill in the art may find useful. Such combinations or sub-combinations are intended to be included within the scope of this specification. As such, the claims may be amended to recite any features or aspects expressly or inherently described in, or otherwise expressly or inherently supported by, the present disclosure. Further, Applicants reserve the right to amend the claims to affirmatively disclaim any features or aspects that may be present in the prior art. The various embodiments disclosed and described in this disclosure may comprise, consist of, or consist essentially of the features and aspects as variously described herein.

In the following description, certain details are set forth in order to provide a better understanding of various aspects of the invention that use mass spectrometry profiling for molecular diagnosis of a benign state or malignant state of a cell, group of cells, lesion, tissue sample, or the like. However, the skilled artisan would understand that these aspects may be practiced without these details and/or in the absence of any details not described herein. In other instances, well-known structures, methods, and/or techniques associated with methods of practicing the various aspects may not be shown or described in detail to avoid unnecessarily obscuring descriptions of other details of the various aspects.

This disclosure describes various elements, features, aspects, and advantages of various embodiments of mass spectrometry profiling systems and methods. It is to be understood that certain descriptions of the disclosed embodiments have been simplified to illustrate only those elements, features and aspects that are relevant to a clear understanding of the disclosed embodiments, while eliminating, for purposes of clarity, other elements, features and aspects. Persons having ordinary skill in the art, upon considering the present description of the disclosed embodiments, will recognize that various combinations or sub-combinations of the disclosed embodiments and other elements, features, and/or aspects may be desirable in a particular implementation or application of the disclosed embodiments. However, because such other elements and/or features may be readily ascertained by persons having ordinary skill upon considering the present description of the disclosed embodiments, and are not necessary for a complete understanding of the disclosed embodiments, a description of such elements and/or features is not provided herein. As such, it is to be understood that the description set forth herein is merely exemplary and illustrative of the disclosed embodiments and is not intended to limit the scope of the invention as defined solely by the claims.

As generally used herein, the term “proteome” refers to the entire complement of proteins produced by an organism or biological system, including modifications made to particular proteins. The proteome of an organism may vary with time, and may also depend on the various stresses that the organism or biological system undergoes. As generally used herein, the term “proteomic profile” refers to information about the protein content of a sample, as characterized by peaks in its mass spectrum corresponding to biomarkers such as proteins, glycoproteins, glycopeptides, peptidoglycans, and other biological substances making up the proteome. The proteomic profile may include all or part of the information, for example, mass spectrometric data, such as m/z values of peaks in the mass spectrum. The proteomic profile may comprise peptide peaks from enzymatic digestion. The proteomic profile may comprise peptidoglycans and/or glycans from enzymatic digestion. For example, the proteomic profile may comprise all or substantially all digested proteins and/or lack the entire complement of proteins. It is also possible that substances that are not proteins may be represented in the mass spectrum, for example carbohydrates or lipo-polysaccharides, as well as endogenous peptides, glycoproteins, glycopeptides, peptidoglycans, and other substances as mentioned above. For the sake of simplicity, however, the term “proteomic profile” and “molecular profile” may be used interchangeably herein and each being represented by the peaks in the mass spectra.

As generally used herein, the terms “subject”, “individual”, and “patient” are used interchangeably herein and refer to any mammalian subject, particularly humans. Other subjects may include cattle, dogs, cats, guinea pigs, rabbits, rats, mice, horses, and so on. In some cases, the methods of the invention may find use in experimental animals, in veterinary application, and in the development of animal models, including, but not limited to, rodents including mice, rats, hamsters, cats, dogs, and primates.

As generally used herein, the term “diagnosis” refers to the determination as to whether a subject is likely affected by a given disease, disorder or dysfunction. The skilled artisan often makes a diagnosis on the basis of one or more diagnostic indicators, i.e., a biomarker, the presence, absence, or amount of which may be indicative of the presence or absence of the disease, disorder or dysfunction.

As generally used herein, the term “prognosis” refers to a prediction of the probable course and outcome of a clinical condition or disease, disorder or dysfunction or treatment thereof. A prognosis of a patient is usually made by evaluating factors or symptoms of a disease that are indicative of a favorable or unfavorable course or outcome. For example, a prognosis may be based on one or more diagnostic indicators, i.e., a biomarker, the presence, absence, or amount of which may be indicative of the likely course or outcome of a treatment of a clinical condition or disease, disorder or dysfunction. It is understood that the term “prognosis” does not necessarily refer to the ability to predict the course or outcome with 100% accuracy. Instead, the skilled artisan will understand that the term “prognosis” refers to an increased probability that a certain course or outcome will occur; that is, that a course or outcome is more likely to occur in a patient exhibiting a given condition when compared to patients not exhibiting the condition.

As generally used herein, the term “tumor” refers to any neoplastic cell growth and proliferation, whether malignant or benign, and all pre-cancerous and cancerous cells and tissues. The terms “cancer” and “cancerous” refer to or describe the physiological condition in animals that is typically characterized in part by unregulated cell growth. Cancer refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has typically not penetrated into the lymphatic or blood vessel system or to tissues other than the primary site. The term “‘locally advanced cancer” describes non-metastatic cancer that has spread to nearby tissues or lymph nodes but not throughout the body. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but it can also refer to a Stage II cancer or a substage of a Stage II cancer. The skilled artisan will appreciate that the classification of a Stage II cancer as either an early stage cancer or a late stage cancer may depend on the particular type of cancer.

As generally used herein, the terms “sample” refers to a sample that may comprise tumor material obtained from a patient. The term encompasses clinical samples, for example, tissue obtained by surgical resection and tissue obtained by biopsy, such as for example a core biopsy or a fine needle biopsy. The term also encompasses samples comprising tumor cells obtained from sites other than the primary tumor, e.g., metastases and circulating tumor cells, as well as well as preserved samples, such as formalin-fixed, paraffin-embedded samples or frozen samples. The term encompasses cells that are the progeny of the patient's tumor cells, e.g., cell culture samples or cell lines derived from primary tumor cells or circulating tumor cells. The term encompasses samples that may comprise protein or nucleic acid material shed from tumor cells in vivo, e.g., bone marrow, blood, lymph, plasma, serum, and the like. The term also encompasses samples that have been enriched for tumor cells or otherwise manipulated after their procurement and samples comprising polynucleotides and/or polypeptides that are obtained from a patient's tumor material.

As generally used herein, the term “treatment” refers to administering an agent, or carrying out a procedure (e.g., radiation, a surgical procedure, etc.) to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a cancer) and/or adverse effect attributable to the disease or condition. These terms also include any treatment of a condition or disease in a mammal, particularly in a human, and include: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; (c) relieving the disease, i.e., causing regression of the disease; (d) reducing the severity of a symptom of the disease and/or (e) reducing the frequency of a symptom of the disease or condition.

The present invention is directed to more efficient and/or cost-effective mass spectrometry profiling systems and methods of making and using the same are described.

Mass spectrometry profiling according to the present invention may be used for molecular diagnosis of a benign state or a malignant state of a cell, group of cells, blood, lymph, skin lesion, tissue sample, or the like. The sample may be obtained from a skin lesion, a primary tumor, a lymph node, or a local or distant metastasis.

The present invention is directed to melanoma diagnostic applications that classify at least three different subtypes of benign nevi and seven different subtypes of melanoma, as well as metastatic melanoma. In the diagnosis of melanocytic lesions, approximately 14% of lesions may be classified as ambiguous based on histopathological review. Additionally, there may be a high degree of discordance among experienced dermatopathologists, both in a diagnosis of benign or malignant as well as identifying the subtype. The present invention includes common subtypes of melanoma (e.g., superficial spreading, nodular, lentigo maligna), those that may be the most challenging to diagnoses (e.g., Spitzoid melanoma/Spitz nevus), as well as other types that may be seen by dermatopathologists and be difficult to diagnose (e.g., desmoplastic, nevoid, acral). The present invention includes conventional and acral, along with Spitz nevi to address the vast majority of melanocytic lesions. The ability of one mass spectrometry classification method to accurately classify/diagnose so many subtypes of melanomas and nevi is unexpected compared to conventional methods that only evaluate Spitzoid lesions. Without wishing to be bound to any particular theory, the present invention may comprise additional subtypes, e.g., blue nevus, blue nevus-like melanoma, and pigmented epithelioid melanocytomas, and/or the location of the tumor, e.g., ocular, anorectal, subungual, and mucosal melanoma, to provide increased utility to the melanoma diagnostic application.

As generally used herein, the term “primary tumor” refers to a malignant tumor (also referred to as cancer) at a first site (i.e., in a first organ or part of the body). In general, when an area of cancer cells at the originating site become clinically detectable, it is referred to as a primary tumor. Some cancer cells also acquire the ability to penetrate and infiltrate surrounding normal tissues in the local area, forming a new tumor. The newly formed “daughter” tumor in the adjacent site within the tissue is called a local metastasis while the formation of a new tumor in a non-adjacent site is called a distant metastasis.

Cancer cells and tumors may be characterized by having different proteins, and different protein expression levels, and/or different forms of proteins than exist in complementary benign cell or tissue samples. Frequently, proteins exist in a sample in a plurality of different forms characterized by detectably different masses. These forms can result from either or both of pre- and post-translational modification. Pre-translational modified forms include allelic variants, splice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation.

When detecting or measuring a protein in a sample, the ability to differentiate between these different forms depends upon the nature of the difference and the method used to detect or measure the difference. For example, an immunoassay using a monoclonal antibody will detect all forms of a protein containing the epitope and will not distinguish between them. In diagnostic assays, this inability to distinguish different forms of a protein diminishes the power of the assay. Further, only known biomarkers may be targeted. Thus, it is useful to employ an assay method that distinguishes between forms of a protein and that specifically detects and measures a desired form or forms of the protein. Distinguishing different forms of an analyte or specifically detecting a particular form of an analyte is referred to as “resolving” the analyte.

The present disclosure describes non-invasive and/or minimally invasive methods that may be used for differentiating between benign melanocytic nevi and malignant melanomas. The methods may include determining whether a proteomic profile of a sample from a patient includes at least one characteristic predictive of one of benign melanocytic nevi and malignant melanomas.

The present invention is generally directed to mass spectrometry imaging and analysis that may be useful in cases that are histologically equivocal or ambiguous, and a firm prediction of benign melanocytic nevi and malignant melanomas cannot be made with absolute certainty. Histology guided mass spectrometry (HGMS) profiling, which is a subset of mass spectrometry imaging, allows for targeted analysis of biomolecules in thin tissue sections from specific cells of interest. HGMS may be used to identify molecular profiles predictive of benign melanocytic nevi and/or malignant melanomas.

Mass spectrometry may be a powerful methodology to detect different forms of a protein because the different forms typically have different masses that may be resolved by the technique (mass spectrometry exploits the intrinsic properties of mass and charge). For example, if one form of a protein is a superior biomarker for a disease over another form of the protein, mass spectrometry may be able to specifically detect and measure the useful form where traditional immunoassay fails to distinguish the forms and fails to specifically detect the useful biomarker.

Mass spectrometry imaging may provide one or more of the following advantages: mass spectrometry works in formalin-fixed paraffin embedded tissue sections; mass spectrometry may provide information to clarify the diagnosis of ambiguous cases by histology; and mass spectrometry is relatively objective, fast, and inexpensive.

The present invention includes a method of differentiating benign cells or tissues from malignant cells or tissue using mass spectrometry analysis. Traditional quantitative mass spectrometry has used electrospray ionization (ESI) followed by tandem mass spectrometry (MS/MS), while newer quantitative and qualitative methods are being developed using matrix assisted laser desorption/ionization (MALDI) followed by time of flight (TOF) mass spectrometry (MS).

In ESI tandem mass spectrometry (ESI/MS/MS), analysis of both precursor ions and product ions is possible, thereby monitoring a single precursor product reaction and producing a signal only when the desired precursor ion is present. Protein quantification has been achieved by quantifying tryptic peptides with the use of isotopically labelled peptide standards. Secondary ion mass spectrometry (SIMS) uses ionized particles emitted from a surface for mass spectrometry at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge.

Laser desorption mass spectrometry (LD-MS) involves the use of a pulsed laser, which induces desorption of sample material from a sample site effectively. This method may be used in conjunction with a mass spectrometer, and can be performed simultaneously with ionization if one uses the right laser radiation wavelength. When coupled with Time-of-Flight (TOF) measurement, LD-MS is referred to as LDLPMS (Laser Desorption Laser Photoionization Mass Spectrometry). The LDLPMS method of analysis gives instantaneous volatilization (fragmentation) of the sample which permits rapid analysis without any wet extraction chemistry. Signal intensity, or peak height, is measured as a function of travel time. The applied voltage and charge of the particular ion determines the kinetic energy, and separation of fragments is due to the different masses causing different velocities. Each ion mass will thus have a different flight-time to the detector. Other ionization techniques include rapid evaporative ionization mass spectrometry (REIMS) and desorption electrospray ionization (DESI) mass spectrometry.

MALDI-TOF mass spectroscopy can quantify intact proteins in biological tissue and fluids, and is thus applicable to direct analysis of biological tissues and single cell organisms with the aim of characterizing endogenous peptide and protein constituents. Quantification by MALDI-TOF mass spectrometry may be absolute or relative in nature, and it may require the use of internal standards and/or corrections for matrix effects, ionization efficiency, and suppressive effects. The sample is generally mixed with a matrix material which facilitates desorption and ionization of the sample. The mass-to-charge ratio (m/z) is measured as a function of travel time, where separation of the fragments is due to the different masses causing different velocities through the drift space and different times of flight to the detector.

Analysis of benign and malignant cells or tissue samples using mass spectrometry provides different protein profiles. Analysis of the protein profiles from known benign and known malignant samples may be used to generate proteomic signatures for each, which may be dependent on the type of tissue and the type or severity of the malignancy. These proteomic signatures may then be used to predict or classify an unknown sample. For example, the proteomic profile of an unknown sample may be compared to reference profiles from one or more reference samples to classify the unknown sample as one of a benign melanocytic nevi and malignant melanoma.

Thus, the present invention provides a method of differentiating benign cells or tissues from malignant cells or tissue using mass spectrometry analysis, where the method generally comprises (a) treating a skin lesion sample; (b) subjecting the skin lesion sample to mass spectrometry; (c) generating a mass spectrometry profile from the skin lesion sample; (d) comparing the mass spectrometry profile of the skin lesion sample to a mass spectrometric proteomic profile of reference samples, wherein the reference profile includes a statistical average profile from a plurality of known benign melanocytic nevi and/or a plurality of known malignant melanoma; and (e) classifying the skin lesion sample as a benign melanocytic nevus or a melanocytic malignant melanoma based on a level of similarity between the mass spectrometry profile of the skin lesion sample and the reference profile. The reference sample may comprise a sample from a patient having a known clinical outcome, e.g., benign melanocytic nevi and malignant melanomas. The reference sample may comprise a sample from a patient having an outcome established by multiple concordant diagnoses by dermatopathologists, e.g., having 2, 3, or more dermatopathologists concur on the diagnostic state of a patient based on review of existing clinical, histopathological, and/or molecular diagnostics data). The reference sample may be used to generate the reference profile. For example, the reference profile may be generated from one or more reference samples, such as at least 10, 25, 50, 100, or more reference samples.

The present invention further describes the use of mass spectrometry imaging methods to differentiate between benign melanocytic nevi and malignant melanomas. Benign melanocytic nevus (pl. nevi), also known as a mole, is the medical term for sharply circumscribed and chronic lesions of the skin, commonly named birthmarks or beauty marks. While Nevi are benign by definition, 25% of malignant melanomas arise from pre-existing nevi.

Skin cancer is the most common form of cancer. There are two major types of skin cancer, keratinocyte cancers (basal and squamous cell carcinomas) and melanoma (malignant melanocytes). Although melanoma accounts for only 3 percent of the skin cancers, it is responsible for greater than 75% of skin cancer deaths and is the seventh most common malignancy in the United States. The National Cancer Institute estimates that 96,480 new cases of melanoma will be diagnosed in the United States in 2019.

Among cells composing skin, melanin-pigment-producing cells are referred to as pigment cells or melanocytes. When these melanocytes become cancerous, a malignant melanoma is developed (i.e., skin lesion). Thus, as used herein, the term “malignant melanoma” may refer to a type of skin cancer involving melanocyte cells. The term “melanoma cells”, as used herein, may refer to melanocytes that have become cancerous. Melanocytes, including melanoma cells, are well known to the skilled person and can be easily identified in a sample due to their location in the stratum basal of the epidermis as well as via melanocyte-specific markers including, but not limited to, Melan-A, HMB45, Protein S100, DCT and TRP2.

TABLE A

| Lesion Depth | 5 year survival rate |
|---|---|
| In situ | 100% |
| <0.75 mm | 99% |
| 0.75 mm-1.49 mm | 95% |
| 1.5 mm-2.49 mm | 82-86% |
| 2.5 mm-3.99 mm | 67-76% |
| >4.0 mm | 54-61% |

In general, the most important prognostic variable in malignant melanoma is the depth of the lesion or tumor (see Table A). Malignant melanoma is staged according to the severity of the disease, into stage 0 to stage 4, measured as a combination of the depth of the lesion or tumor and spread beyond the originating lesion. For example, stage 0 refers to melanoma in situ with 99.9% survival, the melanoma lesion generally having a lesion thickness of less than 0.75 mm. Stage I/II refers to invasive melanoma with 85 to 99% survival, the melanoma lesions generally having a lesion thickness of less than 2 mm. Stage II refers to high risk melanoma with 40 to 85% survival, and can be further divided into T2b (1.00 to 2.00 mm primary tumor thickness, with ulceration), T3a (2.00 to 4.00 mm primary tumor thickness, without ulceration), T3b (2.00 to 4.00 mm primary tumor thickness, with ulceration), T4a (4.00 mm or greater primary tumor thickness without ulceration) and T4b (4.00 mm or greater primary tumor thickness with ulceration). Stage III refers to local metastasis with 25 to 60% survival, and is characterized by positive lymph nodes. Finally, stage IV refers to distant metastasis with only 9 to 15% survival.

The standard care for a lesion suspected to be a malignant melanoma is excisional biopsy with 1-3 mm margins and re-biopsy if the sample is inadequate for diagnosis. In the United States, 1 to 2 million biopsies are performed each year to diagnose melanoma. Of these, 25% cannot be definitively classified using routine histopathology (Am J Surg Pathol, 2009, 33:1146-56), often leading to a misdiagnosis. This high rate of misdiagnosis is problematic on many levels. The false positives lead to unnecessary costly medical interventions (e.g., CT, MRI, or PET scans; additional biopsy), while the false negatives mean increased likelihood of a future presentation with more severe disease and risk of death.

Further, there can be considerable disagreement among dermatologists in the diagnosis of melanocytic lesions. In a 1996 study, 8 pathologists reviewed the same 37 slides containing thin sections of tissue prepared from biopsied nevus: 13 of the slides had complete diagnostic agreement from the 8 pathologists, 10 slides had one discordant diagnosis, 6 slides had 2 discordant diagnoses, and 8 slides had 3 or more discordant diagnoses.

The present invention provides improved methods for differentiating between benign melanocytic nevi and malignant melanomas. More specifically, the present invention includes a targeted approach in which only discrete areas within a tissue sample are analyzed. Histological staining may also be used to guide the acquisition of mass spectrometry images so that each area analyzed may be enriched for a single cell type. Histological staining may include hematoxylin & eosin, immunohistochemistry staining, fluorescent staining, and/or other standard histological staining techniques. Such analysis may be more conducive to statistical analysis and classification algorithm generation. Further, such methods may provide a biological insight into the classification which is not attainable through standard histological techniques (i.e., disease outcome, improved diagnostics, treatment responses, etc.).

Archival formalin-fixed, paraffin embedded tissue samples from benign melanocytic nevi and primary cutaneous malignant melanomas were analyzed by mass spectrometry imaging to identify proteomic differences (i.e., define a proteomic signature). A teaching set comprising 25 nevi and 25 melanomas, was used to generate statistical correlation models using a machine-learning algorithm, wherein the algorithm comprises one of a machine learning algorithm including: Genetic algorithm, Support vector machine, Linear discriminant analysis, Random Forest, Bayesian classifier, Rule-based learners, Decision trees, Artificial neural networks, K nearest neighbors, Naïve Bayes.

A genetic algorithm is a machine-learning algorithm that is similar to classical genetics. Initially, peaks from the spectra may be formed into groups (individuals) and evaluated for how well they may differentiate between the phenotypes (benign and malignant). The ones that perform poorly may be discarded while the best may be bred to form a new generation of offspring. The offspring may be then evaluated for their robustness and the best move on to the next generation. Additionally, mutations may be introduced to the sets of peaks at a user-defined rate to increase “genetic” variability. A maximum number of generations is set that the genetic algorithm is allowed to run over, although, this is often not reached as the calculations are terminated when a local optimum is obtained.

For the genetic algorithm, a combination of peaks that separates best between benign nevi and malignant melanomas was established. A diagnosis of either nevus or melanoma was rendered on a separate validation set of 26 nevi and 29 melanomas based on the proteomic signature, which diagnosis was then correlated with the histopathologic diagnosis. Using the genetic algorithm, mass spectrometry imaging classified 28 of the 29 cases of malignant melanomas, and 22 of the 26 cases of benign melanocytic nevi correctly. The sensitivity for recognizing melanomas was 97% and the specificity was 85%. The teaching set and validation may include samples comprising a random mixture of a limited number of subtypes of cutaneous melanocytic lesions.

A linear discriminant analysis is a machine-learning algorithm that may be used to dimensionally reduce a dataset onto a lower-dimensional space. In a linear discriminant analysis, the computer program is taught to recognize a total spectrum of peaks from the benign samples and the malignant samples. The total spectrum is used as a fingerprint for a diagnosis. Each new spectrum from a new sample is compared to these fingerprints and matched to the one to which it is more similar. By comparison, a genetic algorithm quantifies only a small subset of peaks from the spectrum that best differentiates the groups. All other peaks and parts of the spectra are ignored.

For the linear discriminant analysis, the teaching set comprising 111 nevi and 100 melanomas was used, and a 10% leave-out repeated random sub-sampling cross validation was employed to evaluate accuracy, which was found to be 100%. An optimized classifier was applied to a validation set of 257 nevi specimens and 288 melanoma specimens, where the mass spectrometry results were compared to the known patient diagnoses. The methods of the present invention showed a 99.65% validation accuracy for benign samples in the validation set, and 98.76% validation accuracy for malignant samples in the validation set. The teaching set and validation included samples comprising the most common subtypes of cutaneous melanocytic lesions at the time, i.e., seven (7) melanoma subtypes (acral lentiginous, desmoplastic, lentigo maligna, nevoid, nodular, Spitzoid, and superficial spreading), three (3) benign nevus subtypes (acral, conventional, and Spitz), and metastatic melanoma.

The use of a linear discriminant analysis allows for an accurate differentiation between benign and malignant melanocytic lesions. Applying the classifier developed using the linear discriminant analysis resulted in >99% classification accuracy, which was effective at diagnosing histologically difficult cases. Further, using traditional LC-MS/MS, several specific proteins were identified to be differentiated in the malignant melanocyte lesions (see Table B).

TABLE B

| | Wilcoxon | Area under | |
|---|---|---|---|
| m/z | p-value | ROC Curve | Putative ID |
| 1198.76 | 3.18E−12 | 0.9631 | Actin |
| 976.50 | 4.77E−09 | 0.8916 | Actin |
| 1489.90 | 2.78E−08 | 0.8673 | Trinucleotide repeat- |
| | | | containing gene 6C protein |
| 1399.78 | 2.78E−08 | 0.8679 | Tropomyosin |
| 1501.80 | 3.80E−08 | 0.8679 | Myosin |
| 1646.88 | 6.35E−08 | 0.8562 | Vitronectin |
| 1072.07 | 1.11E−07 | 0.8465 | Collagen alpha-1 |
| 1254.69 | 1.64E−07 | 0.8405 | Vimentin |

m/z - mass/charge ratio;
ROC is receiver operating characteristic

Thus, mass spectrometry imaging may be used to differentiate between benign melanocytic nevi and malignant melanomas in formalin-fixed, paraffin-embedded tissue sections based on proteomic differences. Mass spectrometry imaging may be an objective and reliable method that may be helpful in difficult cases, in which rendering a firm diagnosis of either benign nevus or malignant melanoma may be very difficult. The identification of protein expression profiles, which discriminate between benign melanocytic nevi and malignant melanomas, may lead to the discovery of clinically useful protein biomarkers and translate into tumor biomarkers that may be incorporated into standard diagnostic and treatment strategies.

Annotation of the sample may occur after part of or the entire sample has been subjected to mass spectrometry such that selection of the areas of interest is performed post-acquisition of mass spectral data. In this example, the analyzed sample may be stained, and the digital microscopy image annotated for regions of interest; these regions of interest can be superimposed onto the image of the sample use in the mass spectrometry analysis to isolate their components of the spectral data file. In another example, a serial section of the sample may be stained, and the digital microscopy image annotated for regions of interest; these regions of interest can be superimposed onto the image of the sample used in the mass spectrometry analysis to isolate their components of the spectral data file.

The method may comprise collecting a first serial section of the sample and a second serial section of the sample, staining the first serial section of the sample, and subjecting the second serial section of the sample to mass spectrometry. For example, a first serial section of the sample may be stained and a second serial section may be subjected to mass spectrometry. In other words, the treated sample may not be subjected to mass spectrometry. Instead, the regions of the other one of the serial sections that correspond to the annotated sections of the treated sample may be subjected to mass spectrometry.

Referring to FIG. 7, a method of differentiating benign melanocytic nevi from malignant melanomas may generally comprise (a) treating a skin lesion sample (100) from a patient; (b) subjecting the skin lesion sample (100) to mass spectrometry (110); (c) generating a mass spectrometry profile from the skin lesion sample (120); (d) comparing the mass spectrometry profile of the skin lesion sample to a mass spectrometric proteomic profile of reference samples (130), wherein the reference profile includes a statistical average profile from a plurality of known benign melanocytic nevi and/or a plurality of known malignant melanoma; and (e) classifying the skin lesion sample (140) as a benign melanocytic nevus or a melanocytic malignant melanoma based on a level of similarity between the mass spectrometry profile of the skin lesion sample and the reference profile.

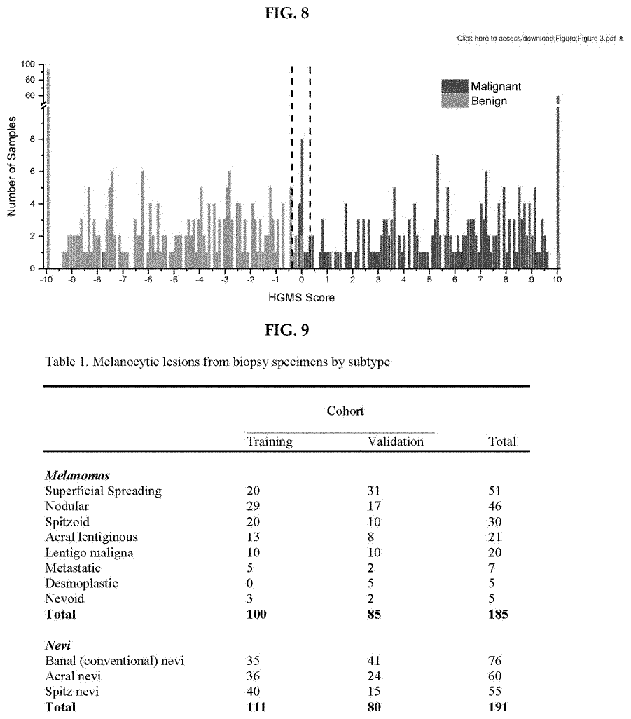

Referring to FIGS. 7 and 8, the method may comprise treating the sample prior to and/or after subjecting the sample to mass spectrometry. Treating a sample may comprise at least one of collecting at least one serial section of the sample, staining the sample, subjecting the sample to deparaffinization and antigen retrieval, subjecting the sample to on-tissue tryptic digestion, applying a MALDI compatible matrix to the sample, annotating a digital microscopy image of the reference profile and imposing the annotated digital microscopy image of the reference profile onto an image of the sample. For example, the method may comprise subjecting the sample to deparaffinization, subjecting the sample to antigen retrieval, subjecting the sample to on-tissue tryptic digestion, and/or applying a MALDI compatible matrix to the sample prior to subjecting the sample to mass spectrometry. For example, the method may comprise staining the sample prior to subjecting the stained sample to mass spectrometry. In another example, the method may comprise staining the sample after subjecting the sample to mass spectrometry. Annotating the sample may comprise marking targets, or areas of interest on the sample that may have a diameter from 10-500 micrometers, such as 50-100 micrometers and 250-350 micrometers. Subjecting the sample to mass spectrometry may comprise subjecting the targets or areas of interest to mass spectrometry. Subjecting the sample to mass spectrometry may comprise subjecting the entire sample, i.e., targets or areas of interest as well as other areas of the sample, to mass spectrometry.

The method may have a sensitivity and a specificity of at least 95% in correctly classifying the skin lesion sample, such as at least 96%, at least 97%, at least 98%, at least 99%, or even at least 99.5%.

The method may comprise repeating the steps of (a)-(e) on the skin lesion, such as, for example, in 6-12 months, 6-18 months, 6-24 months, 12-18 months, 12-24 months, 18-24 months, or longer when the patient is identified as having benign melanocytic nevi.

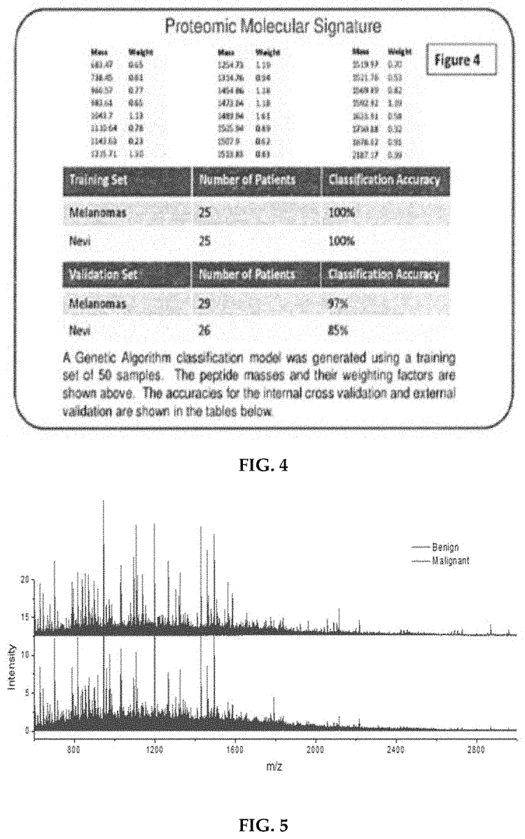

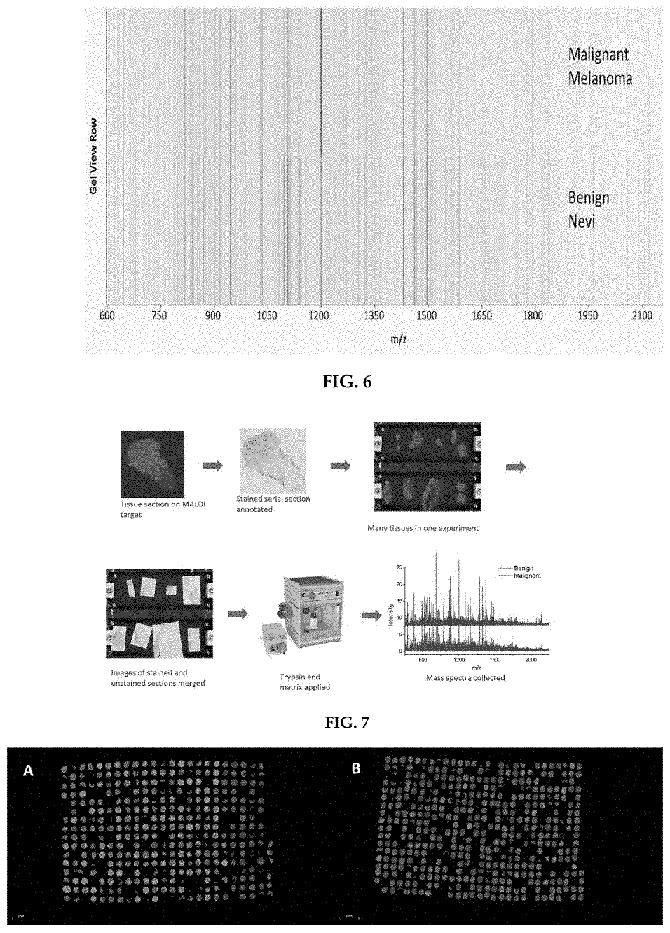

The classification may comprise use of a genetic algorithm or a linear discriminant analysis to generate a proteomic signature of a reference sample. The reference sample may include a proteomic signature of a known benign sample (i.e., cell, lesion, tumor) and a known malignant sample (i.e., cell, lesion, tumor). The mass spectrometric profile may comprise analysis of a specific subset of markers as defined by the genetic algorithm. The mass spectrometric profile may comprise an average spectrum from a known benign sample (FIG. 5, top) and an average spectrum from a known malignant sample (FIG. 5, bottom).

The mass spectrometry may comprise secondary ion mass spectrometry, laser desorption mass spectrometry, matrix assisted laser desorption mass spectrometry, electrospray mass spectrometry, desorption electrospray ionization, or laser ablation electrospray ionization mass spectrometry.

The method may comprise generating the sample from the patient.

The method may comprise making a treatment decision for the patient.

The method may comprise treating the patient with at least one of chemotherapy, immunotherapy, toxin therapy, surgery, and radiotherapy when the patient is identified as having malignant melanoma.

The method may comprise assessing one or more patient history parameters from the patient.

The method may comprise performing histologic analysis on the sample.

The method may comprise performing a mass spectrometric analysis of a known benign melanocytic nevus and/or melanocytic malignant melanoma lesion.

The method may comprise making a prediction of the patient's survival based on the classification.

The method may comprise determining a form of treatment, e.g., selecting a drug, based on the classification.

In various embodiments, the skin lesion sample comprises one of melanocytic components and stromal components. The melanocytic components may be confined to the area of the lesion. The method may comprise examining melanocytic and/or stromal components of the skin lesion sample. The method may comprise examining the normal (stromal) tissue directly adjacent and/or proximal to the skin lesion sample. The method may analyze the proteomic changes in the tissue microenvironment that may be indicative of a diagnosis or prognosis.

The statistical average profile of the method may be generated using a genetic algorithm which defines a mass spectrometric profile comprising one or more markers.

In various embodiments, the mass spectrometric profile may comprise one or more markers. In various embodiments, the mass spectrometric profile may comprise one or more peptide peaks at one or more of m/z 683.47±0.2, m/z 738.45±0.2, m/z 960.57±0.2, m/z 983.61±0.2, m/z 1043.70±0.2, m/z 1110.64±0.2, m/z 1143.63±0.2, m/z 1215.71±0.2, m/z 1254.73±0.2, m/z 1314.76±0.2, m/z 1454.86±0.2, m/z 1473.84±0.2, m/z 1489.94±0.2, m/z 1505.94±0.2, m/z 1507.90±0.2, m/z 1513.83±0.2, m/z 1519.97±0.2, m/z 1521.76±0.2, m/z 1569.89±0.2, m/z 1592.92±0.2, m/z 1633.91±0.2, m/z 1730.88±0.2, m/z 1878.02±0.2, and m/z 2187.17±0.2. In various embodiments, the mass spectrometric profile may comprise one or more peptide peaks at m/z 976.50±0.2, m/z 1072.07±0.2, m/z 1138.61±0.2, m/z 1198.76±0.2, m/z 1254.69±0.2, m/z 1399.78±0.2, m/z 1489.90±0.2, m/z 1501.80±0.2, and m/z 1646.88±0.2.

The method may comprise using the mass spectrometric profile to determine which form of treatment may be more therapeutically effective for the patient. In various embodiments, the form of treatment may comprise a drug compound dosage regimen, antibody-drug conjugate dosage regimen, radiochemical compound dosage regimen, radiation therapy, and/or surgical excision,

The method may comprise immunohistochemical analysis on the skin lesion sample. In various embodiments, the patient may previously have had immunohistochemical analysis of the lesion. In various embodiments, the previous immunohistochemical analysis indicated that the lesion was a benign melanocytic nevus. In various embodiments, the previous immunohistochemical analysis indicated that the lesion was a malignant melanoma.

The statistical average profile of the method may be generated using a linear discriminant analysis on the total spectrum from the plurality of known normal tissue, the plurality of known benign melanocytic nevi, and/or the plurality of known malignant melanoma. The linear discriminant analysis may show a sensitivity and a specificity of at least 95% in correctly classifying the skin lesion sample.

The plurality of known normal tissue, the plurality of known benign melanocytic nevi, and the plurality of known malignant melanoma may be classified by immunohistochemical analysis, genetic analysis, patient clinical outcome, or a combination thereof.

Without wishing to be bound to any particular theory, subjecting the sample to mass spectrometry prior to annotation may reduce the reproducibility of results for one or more of the following reasons: exposure to the laser may cause mild to moderate destruction of the sample which may impede proper observation and analysis of the sample for staining and annotation; the use of larger regions of the sample for analysis may encourage less particularity when defining the region for cancer cell analysis; and decreased particularity may result in a wider array of cell types within the sample increasing the variability of results. Limiting the data to the areas of interest may provide for more accurate classification and results as there is less “noise” from inappropriate cell types. Noise, in this context, consists of signals originating from other cell types than the ones of interest. This may include red blood cell, inflammatory cells, and the like.

The mass spectrometric proteomic profile may include one or more peaks of monoisotopic mass. The intensity of or area under the peak for the monoisotopic mass may be used to determine the abundance value. The monoisotopic mass is the sum of the masses of the atoms in a molecule using the unbound, ground-state, rest mass of the principal (most abundant) isotope for each element instead of the isotopic average mass. Monoisotopic mass is typically expressed in unified atomic mass units (u), also called Daltons (Da). The mass spectrometric proteomic profile may not include all of the peaks detected from the sample. Without wishing to be bound by theory, it is thought that the peaks represent unique biomarkers that are useful for fast detection and identification of cancer in the samples or biomarkers that are useful for determining prognosis for a patient. These biomarkers do not need to be identified (e.g., myosin) to be used as mass signature components of the mass spectrometric proteomic profile. When samples that have been enzymatically digested as part of the treatment process, more than one peak may be associated with the same biomarker as a result of the fragmentation of the protein parent molecules into constituent sub-protein level biomolecules (e.g. peptides, glycans). For example, one protein biomarker may have several peptides detected and comprising part of the mass spectrometric proteomic profile.

As discussed above, a diagnosis of benign nevi and malignant melanomas may be based on characteristic similarities or differences (e.g., proteomic signature) between tumor samples and/or a tumor sample and reference sample. For example, if the proteomic profile is presented in the form of a mass spectrum, the proteomic signature may be a peak or a combination of peaks that differ, qualitatively or quantitatively, from the mass spectrum of a corresponding reference sample. Thus, the appearance of a new peak or a combination of new peaks in the mass spectrum, or any statistically significant change in the amplitude or shape of an existing peak or combination of existing peaks, or the disappearance of an existing peak in the mass spectrum may be considered a proteomic signature for one of a benign nevus or a malignant melanoma.

Statistical methods for comparing proteomic profiles may be defined by the peak amplitude values at key mass/charge (m/z) positions along the horizontal axis of the mass spectrum. A characteristic proteomic profile may, for example, be characterized by the pattern formed by the combination of spectral amplitudes at given m/z vales. The presence or absence of a proteomic signature for one of a benign nevus or a malignant melanoma, or the substantial identity of two proteomic profiles, may be determined by matching the proteomic profile of a tumor sample with the proteomic profile of a reference sample using an appropriate algorithm.

The presently disclosed invention provides improved methods for differentiating between benign melanocytic nevi and malignant melanomas. More specifically, the present invention includes a targeted approach in which only discrete areas within a tissue sample are analyzed. Histological or immunohistological staining may also be used to guide the acquisition of mass spectrometry imaging so that each area analyzed may be enriched for a single cell type. Such analysis may be more conducive to statistical analysis and classification algorithm generation. Further, such methods may provide a biological insight into the classification which is not attainable through standard histological techniques (i.e., disease outcome, improved diagnostics, treatment responses, etc.). The present invention may include samples representing multiple subtypes of benign melanocytic nevi and malignant melanomas, as well as samples representing metastatic melanoma in both the training and validation sets, which have been collectively shown to provide an increase in the overall accuracy of a classification algorithm and model and increase in its utility for evaluating samples of unknown subtype.

According to the present invention, a tissue sample is collected onto an indium tin oxide (ITO) coated glass slide that is compatible with a MALDI TOF mass spectrometer. A serial section is collected onto a standard microscopy slide and stained with hematoxylin and eosin (H&E). The stained section is scanned using a digital slide scanner and made available to a pathologist who annotates histological regions of interest within the section. The annotated stained section is registered with a digital image of the serial unstained section using Photoshop® (or equivalent software) allowing for the coordinates of the annotations to be obtained. The unstained section is subjected to sample preparation including deparaffinization, antigen retrieval, tryptic digestion, and matrix application. Mass spectra are collected from the designated locations and the spectra subjected to statistical analysis.

A biomarker in the tissue sample may be modified before analysis to improve its resolution or to determine its identity. For example, the biomarkers may be subject to proteolytic digestion before analysis using a protease. Proteases, such as trypsin, PNGaseF, and GluC, that are likely to cleave the biomarkers into a discrete number of fragments may be particularly useful. Similarly, heat-induced digestion and chemical cleavage can generate reproducible peptide fragments from proteins. Due to their individual characteristics, each fragment will be detected as a unique mass and will experience differences in ionization efficiency, resolution, and sensitivity during mass spectrometry analysis. One, multiple, or all fragments of a biomarker may be detected in a single analysis. The fragments that result from digestion function as a fingerprint for the biomarkers, thereby enabling their detection indirectly. This may be particularly useful where there are biomarkers with similar molecular masses that might be confused for the biomarker in question. Also, proteolytic fragmentation may be useful for high molecular weight biomarkers because smaller biomarkers may be more easily resolved by mass spectrometry. Typically, the peptide fragments resulting from the enzymatic digestion of a protein biomarker are more easily detected than the original parent protein itself due to improved ionization efficiency and desorption efficiency, leading to improved sensitivity. Additionally, proteolytic digestion allows for measurement of uncrosslinked protein segments from larger proteins that have been crosslinked due to fixation processes. This is particularly useful in FFPE tissues where large protein biomarkers are crosslinked and very difficult to liberate from the tissue surface through desorption ionization processes. Carefully controlled enzymatic digestion treatment that maintains spatial localization increases the sensitivity of detection for the resultant biomarker fragment peptides, glycans, in a robust and reproducible manner. In a histology guided mass spectrometry approach, this sample treatment process results in complex biomarker fragment spectra for each type of cell subpopulation, for which these types of large, robust data sets are well-suited for statistical analysis by machine learning and artificial intelligence platforms. In another example, biomarkers may be modified to improve detection resolution. For instance, neuraminidase may be used to remove terminal sialic acid residues from glycoproteins to improve binding to an anionic adsorbent and to improve detection resolution. In another example, the biomarkers may be modified by the attachment of a tag of particular molecular weight that specifically binds to molecular biomarkers, further distinguishing them.

### EXAMPLES

The present invention may be better understood when read in conjunction with the following representative examples. The following examples are included for purposes of illustration and not limitation.

### Example 1

Tumor Specimens.

A total of 105 archival formalin-fixed, paraffin-embedded tissue samples from 51 histologically unequivocal benign melanocytic nevi (BMN) and 54 conventional primary cutaneous malignant melanomas (MM) were collected. All cases were re-reviewed by a dermatopathologist to confirm the diagnosis. The samples were randomly divided into 2 cohorts: a training set and a validation set. The training set consisted of 25 BMN and 25 MM. The validation set consisted of 26 BMN and 29 MM for the genetic algorithm or 25 BMN and 29 MM for the linear discriminant analysis.

Mass Spectrometry Analysis.

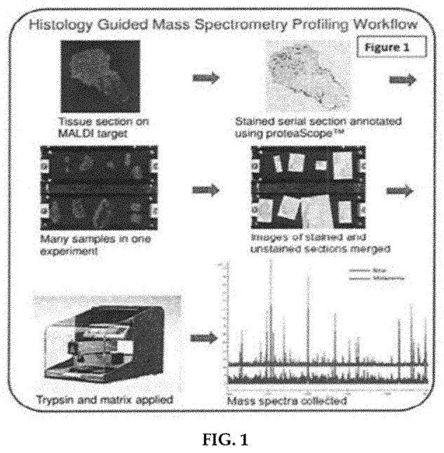

Serial sections, 5 μm thick, were cut from formalin-fixed, paraffin-embedded (FFPE) tissue blocks. One section per sample was mounted onto a conductive glass slide, whereas the consecutive serial section was mounted onto a regular glass slide and stained with hematoxylin and eosin, which served as a reference section (see FIG. 1). Unstained sections were subjected to paraffin removal with xylene and graded ethanol washes. After air-drying, antigen retrieval was performed by heating the sections in TRIS buffer. Antigen-retrieved sections were stored in a desiccator at room temperature (about 22° C. to about 25° C.) until enzyme and matrix deposition for no longer than 2 days.

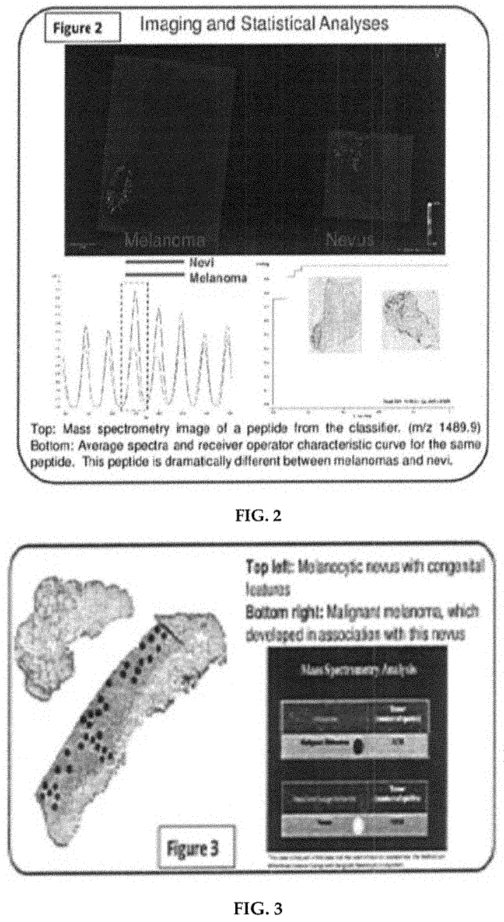

Mass spectral profiles were acquired from the tissue using a histology guided profiling approach as follows. Digital images of the histology slide were acquired using an Olympus VS120 microscope at 20× magnification. The dermatopathologist marked about 100-μm-diameter color-coded areas of interest (about 40 per section) on the digital image of the hematoxylin and eosin stained section. Using image-processing software (e.g., Photoshop), the histology-marked image was merged to an image of the unstained section (FIG. 1). Trypsin was applied to the entire surface of the unstained tissue sections using a SunChrom SunCollect or an HTX M5 robotic reagent sprayer. The digestion was allowed to proceed in a humidified environment for about 4 hours at about 37° C. An α-Cyano-4-hydroxycinnamic acid matrix was applied via the SunCollect or M5 robotic sprayer. The merged annotated images were loaded into the mass spectrometer (ultrafleXtreme or rapifleX, Bruker Daltonics) and after alignment using fiducial points, mass spectra were collected only from the annotated areas (FIG. 2).

Development of Training Models.

The mass spectra profiles were exported and statistical analyses and classification algorithm generation was carried out using: ClinProTools (Bruker) for the genetic algorithm or SCiLS Lab for the linear discriminant analysis. For the analysis, samples were sorted into training, validation, and classification sets.

Statistical analysis of the mass spectrometry data using the genetic algorithm allowed for objective generation of a statistical model. The genetic algorithm comprised 24 peaks, determined by statistical comparison of the peaks in the training set for benign melanocytic nevi and malignant melanomas. The 24 peaks represent different peptides, which are differentially expressed in both groups. These peptides have the following masses: 683.47, 738.45, 960.57, 983.61, 1043.7, 1110.64, 1143.63, 1215.71, 1254.73, 1314.76, 1454.86, 1473.84, 1489.94, 1505.94, 1507.9, 1513.83, 1519.97, 1521.76, 1569.89, 1592.92, 1633.91, 1730.88, 1878.02, 2187.17, (FIG. 4). After these 24 peaks were established from the training set, the cases from the validation cohort were subjected to the algorithm.

Alternatively, or in conjunction with the genetic algorithm generation, hypothesis testing and discriminative m/z value receiver operating characteristics (ROC) analysis was carried out on the training set (see FIG. 4) using a linear discriminant analysis. In this case, over 166 peaks from the full spectral analysis were identified which were significant with Bonferroni correction, 92 of these peaks had areas under the receiver operator characteristics curve >0.8 or <0.2.

After development of a genetic algorithm model or linear discriminant analysis model on the training set, it was applied to an independent validation set.

Patient-Level Characteristics.

The cohort of benign melanocytic nevi (BMN) came from 51 patients, who ranged from 10 to 62 years of age (mean-25), 29 female and 22 male patients. The nevi were distributed on the trunk (31), head and neck (12), upper extremity (6), and lower extremity (2). None of the lesions recurred or metastasized, and all patients are alive with a follow-up ranging from 4 to 11 years (mean, 6.3). The melanoma (MM) cohort comprised 54 patients from 26 to 97 years old (mean, 68), 21 female and 33 male patients. The distribution was as follows: trunk (22), head and neck (12), upper extremity (10), and lower extremity (10). The depth of the MM ranged from 1.0 to 16.0 mm (mean, 4.8 mm). The follow-up ranged from 4 to 24 years (mean, 13).

Results.

Mass spectra from each dotted area (FIG. 3) on each sample from the training set were obtained. Data were analyzed using ClinProTools to develop a GA, or SCiLS Lab for the linear discriminant analysis, as described above, and classification models were built using a training set of biopsies from 25 BMN and 25 MM. After a molecular signature was determined based on data from the training set, it was then tested on a validation cohort.

For the genetic algorithm, the validation cohort was 26 BMN and 29 MM. The method correctly recognized 22 of 26 BMN (85%) and 28 out of 29 MM (97%) in the validation cohort (FIG. 4). Thus, the mass spectrometry imaging method using a genetic algorithm showed a sensitivity of about 97% and a specificity of about 85% in correctly identifying MM based on tumor analysis in the validation set.

For the linear discriminant analysis, the validation cohort was 25 BMN and 29 MM. The method correctly recognized 25 of the 25 BMN (100%) and 28 of the 29 MM (97%). Thus, the mass spectrometry imaging method using a linear discriminant analysis showed a sensitivity of 100% and a specificity of about 97% in correctly identifying MM based on tumor analysis in the validation set.

### Example 2

Distinguishing benign nevi from malignant melanoma using conventional histopathological criteria may be very challenging and represent one of the most difficult areas in Dermatopathology. The present invention identifies proteomic differences that may more reliably differentiate between benign and malignant melanocytic lesions relative to conventional methods. Histology Guided Mass Spectrometry (HGMS) profiling analysis on formalin-fixed, paraffin embedded tissue samples was used to identify in situ differences at the proteomic level between different types of benign nevi and melanomas. A total of 756 cases, of which 357 cases of melanoma and 399 benign nevi, were studied. The specimens originated from both biopsies (376 samples) and tissue microarray cores (380 samples). After generating mass spectra from each sample, classification models were built using a training set of biopsy specimens from 111 nevi and 100 melanomas. The classification algorithm developed on the training data set was validated on an independent set of 288 nevi and 257 melanomas from both biopsies and TMA cores. In the melanoma cohort, 239/257 (93%) cases classified correctly in the validation set, 3/257 (1.2%) classified incorrectly, and 15/257 (5.8%) classified as indeterminant. In the cohort of nevi, 282/288 (98%) cases classified correctly, 1/288 (0.3%) classified incorrectly, and 5/288 (1.7%) were indeterminant. HGMS showed a sensitivity of 98.76% and specificity of 99.65% in determining benign versus malignant (indeterminant classifications were excluded from these calculations). The present invention demonstrated that HGMS proteomic analysis may be an objective and reliable test with minimal tissue requirements, which can be a helpful ancillary test in the diagnosis of challenging melanocytic lesions.

Introduction

Over 4 million skin biopsies are collected annually in the United States due to suspicion of melanoma. Of these, as many as 14% are labeled as indeterminant, based on initial histopathological evaluation of hematoxylin and eosin (H&E) stained sections of the lesions. A high degree of discordance can be observed when different dermatopathologists review the same biopsy specimen. Melanocytic lesions that are labeled as indeterminant are frequently treated as if they were malignant due to risks associated with failure to diagnose. However, significant morbidity associated with wide re-excision, lymph node biopsy and completion lymphadenectomy, toxicity due to therapeutic agents, and emotional stress can occur. In addition, there is unnecessary cost to patients and payers associated with these treatments in cases that were not malignant.

A variety of ancillary tests such as comparative genomic hybridization, fluorescent in situ hybridization, and a test based on a gene expression signature including a set of 23 differentially expressed genes may be helpful.

Histology Guided Mass Spectrometry (HGMS) when applied to melanocytic lesions renders a proteomic profile of the lesional melanocytes in FFPE tissue sections. One of the major advantages of HGMS is that the proteomic content of the lesions provides a molecular snapshot of the active state of the cells, not just the genetic potential for malignancy. A previous study highlighted proteomic differences between Spitz nevi, one type of benign nevus, and Spitzoid malignant melanomas and identified a molecular signature of five peptides that could correctly classify the tumors. The present invention showed HGMS could reliably differentiate between benign nevi and malignant melanoma in sample cohorts containing a wide variety of histological subtypes based on proteomics differences.

Materials and Methods

Sample Cohort

Archival formalin-fixed paraffin-embedded (FFPE) tissue samples of benign nevi and malignant melanomas were used. Sample selection was carried out through queries of tumor bank databases. A total of 756 cases were included in the study. A requirement for each case was to have an unequivocal histopathological diagnosis of benign nevus or malignant melanoma. An inclusion criterion was a follow-up of at least 5 years for all benign cases. Follow-up was shorter in some malignant cases, in which there were adverse events such as death and/or metastatic disease. Samples with insufficient melanocytic component, melanocytic lesions not primary to the skin, re-excision specimens, specimens directly exposed to radiation treatment, and specimens from patients currently, or recently, receiving chemotherapy were excluded from the study.

This study was conducted with institutional review board approval.

A total of 376 biopsy specimens of nevi and melanomas were contributed by experienced dermatopathologists from 7 centers in 4 countries. Additionally, 380 samples, 172 melanomas and 208 nevi from tissue microarray (TMA) cores, were also included in the study.

There were a total of 357 melanomas of which 185 were biopsy specimens and 172 were TMA cores. Seven different histological types of melanoma were studied: superficial spreading, nodular, Spitzoid, acral lentiginous, lentigo maligna, desmoplastic, nevoid, as well as metastatic melanoma. A total of 399 nevi, 191 from biopsy specimens and 208 from TMA cores, were analyzed. The cohort of nevi included conventional, Spitz nevi, and acral nevi. The training set consisted of 211 biopsy specimens only, 100 melanomas and 111 nevi. In the validation set there were 257 melanomas (85 from biopsy specimens and 172 from TMA cores) and 288 nevi (80 from biopsy specimens and 208 from TMA cores) (Table 1). The majority of the melanomas were seen by multiple dermatopathologists at consensus conference at the time of the initial diagnosis. Each case was re-reviewed by two dermatopathologists who were blinded to the diagnosis of the contributing dermatopathologist. Discrepancies were adjudicated in a consensus conference and cases for which there was a discrepancy were excluded. Only cases with an unequivocal consensus diagnosis of nevus or melanoma were included in the study. Clinical and histopathological parameters were recorded for the biopsy cases including patient's age, gender, and primary tumor anatomical location. For the melanoma cases, tumor thickness, presence or absence of ulceration, and mitotic rate were documented in accordance with the American Joint Committee on Cancer (AJCC).

Histology Guided Mass Spectrometry

The histology guided mass spectrometry (HGMS) workflow is summarized in FIG. 7. Briefly, two serial sections for each case, 5-μm thick, were cut from FFPE biopsies specimens. One section per sample was collected onto an indium-tin oxide coated glass slide that was compatible with the mass spectrometer, and the consecutive serial section was mounted onto a regular glass slide and stained with H&E, which served as a reference section. The H&E stained slides were digitized (Huron LE120, Huron Digital Pathology, St. Jacobs, ON, Canada) and uploaded to an online viewing and annotation portal (proteaScope, Protea Biosciences, Inc., Morgantown, W. Va.) for histopathological review. Areas of pure melanocytic component (˜20 per sample, 100 μm diameter) were annotated by a dermatopathologist (RL) on the digital images. A minimum of 5 annotations per sample were required to complete the analysis and classification.

Sections for mass spectrometry were deparaffinized before antigen retrieval in TRIS buffer, pH 9 (Decloaking Chamber™ NxGen, BioCare Medical, Pacheco, Calif.) and buffer exchanged to water. Images of the unstained sections were acquired using a flatbed document scanner. Annotated images of the H&E stained sections were digitally merged with the images of the unstained sections.

Sections for mass spectrometry were subjected to on-tissue tryptic digestion and matrix application using an HTX M5 robotic reagent sprayer (HTX Technologies, LLC, Chapel Hill, N.C.). Mass spectrometry data from designated areas were collected using either a Bruker ultrafleXtreme™ or Bruker rapifleX™ MALDI TOF mass spectrometer (Bruker Daltonics, Billerica, Mass.), which operated in positive ion reflectron mode using FlexImaging software for plate alignment and designation of areas of interest. Data were acquired from the TMAs in their entirety, but only data from the melanocytic component, based on histological review and annotation, was used for statistical analysis. Examples of classification results for two of the six TMAs analyzed are shown in FIG. 8. FIG. 8 shows classification results for nevi and melanoma cases from TMA cores. Each pixel of acquired data is colored red for a malignant classification or green for a benign classification. All cores analyzed in (A) were collected from benign nevi and all cores analyzed in (B) were from malignant melanomas. Only melanocytic component was utilized for the classification. Cores with no data either did not contain any melanocytic component or represented other non-melanocytic tumors or controls.

Statistical Data Analysis and Machine Learning

Data from biopsy specimens were loaded into SCiLS Lab Pro 2019b where they were preprocessed including baseline correction and normalization. Peak integration boundaries were manually defined. Samples were tagged to belong to one of four classification groups: Melanoma Training, Nevi Training, Melanoma Validation, and Nevi Validation. Hypothesis testing was carried out on the samples in the training set. A linear discriminant analysis (LDA) machine learning algorithm was developed using the training set and the accuracy estimated using a leave-10%-out repeated random subsampling internal cross validation. The optimized LDA algorithm was then applied to the validation set. HGMS results were compared to the histopathological diagnoses in each case.

Results

Characterization of the Study Sample

Our cohort of benign nevi from biopsy specimens came from 191 patients, who ranged from 3 to 82 years of age (mean=27.2), 108 females and 83 males. The most common primary anatomical location was the lower extremity (85) followed by the trunk (60), the head and neck region (29) and the upper extremity (17). The sample cohort was comprised of conventional (76), acral (58) and Spitz (57) nevi (Table 2). None of the lesions recurred or metastasized and all patients were alive with a follow-up of 5 to 26 years (mean 9.4). The TMA cohort consisted of cores from 208 benign nevi from patients with a follow-up between 17 and 21 years (mean=18.85). There was no recurrence or adverse events in this group. The melanoma cohort from biopsy specimens comprised patients from 23 to 99 years of age (mean=65), 109 males and 76 females. The distribution in descending order was as follows: trunk (56), lower extremity (54), head and neck (40), and upper extremity (35). Seven histological types of primary melanoma as well as metastatic melanoma were included in the study. They were: superficial spreading (50), nodular (46), Spitzoid (29), acral lentiginous (21), lentigo maligna (22), metastatic melanoma (7), desmoplastic melanoma (5), and nevoid melanoma (5). The tumor thickness of the melanomas ranged from 0.33 to 21.0 mm (mean=3.03). The follow-up was between 0.5 to 21 years (mean=5.86). A summary of the clinical and histopathological characteristics of the melanoma patients is shown in Table 3. The TMA cohort consisted of 172 primary melanomas. Follow-up data, patients' demographics, and information about the histological type for those cases were not available.

Mass Spectrometry Analysis

A total of 756 benign nevi and malignant melanomas were analyzed. The specimens originated from both, tissue biopsies (376 samples) and TMA cores (380 samples). The training set consisted of 211 biopsy samples, 100 melanomas and 111 nevi. A total of 1075 monoisotopic peaks were selected using the average spectrum from the data set. Of these, 879 peaks were found to be statistically significant with p-values<4.65×10−5 after applying a Bonferroni correction. A linear discriminant analysis (LDA) machine learning algorithm was optimized using SCiLS Lab Pro 2019b and an internal cross validation using leave-10%-out repeated random subsampling resulted in 94.1% spectral classification accuracy. The optimized LDA algorithm was applied to an independent set of biopsies and TMA cores of nevi and melanomas to assess the accuracy of the model.

This validation set consisted of 165 biopsy samples (85 melanomas and 80 nevi) and 380 TMA cores (172 melanomas and 208 nevi). Examples of classification results for two of the six TMAs analyzed are shown in FIG. 8. The SCiLS classification output was converted to a numerical score ranging from −10 to +10 that classified the sample as likely benign, indeterminant, or malignant. The ranges for classification of a sample were as follow (FIG. 9): benign (−10 to −0.371), indeterminant (−0.370 to +0.370), and malignant (+0.371 to +10) score ranges. The histogram of HGMS protein profile scores of known benign (green) and malignant samples (red) are also shown.

In the validation set, out of the total of 545 samples, 521 (95.6%) classified correctly; 4 (0.7%) classified incorrectly, and 20 (3.7%) classified as indeterminant. In the melanoma group 239/257 (93.0%) melanomas classified correctly. Only 3 melanomas (1.2%) were classified incorrectly as benign. Two of these were superficial spreading type and one was Spitzoid melanoma. Additionally, 15/257 cases (5.8%) from the melanoma group classified as indeterminant; 7 cases from biopsies and 8 cases from TMA cores.

In the cohort of nevi, 282/288 (97.9%) cases classified correctly in the validation set. Only 1/288 (0.3%) case of a compound Spitz nevus was incorrectly classified.

Additionally, 5/288 nevi (1.7%) from TMA cores classified as indeterminant. The indeterminant samples were excluded from calculations of sensitivity, specificity, and overall accuracy. The sensitivity of the method was 98.76%, and the specificity was 99.65%. The overall accuracy across all specimens with a definitive HGMS diagnosis was 99.24%. The sensitivity of the method was 93.0%, the specificity was 97.9%, and the overall accuracy across all specimens analyzed by HGMS was 95.6% when the indeterminant samples were included in the calculations. These results are summarized in Table 4.

Discussion

Behavior of genome products is difficult to predict from the gene sequence and RNA quantitation, and measurement of gene expression at the protein level is more informative since protein contains information that collectively indicates the actual state rather than the potential functional state of the cell. Molecular profiling by HGMS allows us to look beyond classic histology and objectively analyze melanocytic lesions based on their proteomic content. Previously, we highlighted proteomic differences between the melanocytes of Spitz nevi and Spitzoid malignant melanoma and identified a molecular signature of 5 peptides that could correctly classify the tumors. In a subsequent study, HGMS analysis was performed on diagnostically challenging atypical Spitzoid neoplasms and, when compared and correlated with the clinical behavior, mass spectrometry diagnosis showed stronger association with clinical outcome than did the histopathological diagnosis. The mass spectrometry algorithm was successfully applied in a challenging case of a baby born to a mother who developed melanoma during pregnancy. The baby was born with atypical melanocytic lesions on the trunk. The differential diagnosis for the newborn's lesions included congenital nevi versus metastatic melanoma from the mother. HGMS analysis classified the mother's lesion as malignant melanoma and the baby's lesions as benign nevi. The latter was also supported by the presence of a Y-chromosome in the baby's lesional cells confirming that they were not from the mother. In another case, HGMS was able to correctly differentiate between a proliferative nodule within a giant congenital melanocytic nevus and congenital melanoma in a 2-month-old baby.

In the current study we evaluated 7 histological types of primary melanoma as well as metastatic melanoma and 3 types of benign nevi. We developed a classification algorithm using HGMS profiling and machine learning that could provide proteomic molecular insight to aid in the diagnosis of challenging melanocytic lesions. Our study showed that benign nevi and melanomas could be successfully distinguished using HGMS analysis based on detection of proteomic differences. HGMS was able to differentiate melanomas from nevi with 98.76% sensitivity and 99.65% specificity in the validation cohort with overall accuracy of 99.24% (indeterminant samples were excluded from the calculations). Without wishing to be bound to any particular theory, it is believed that including the 7 histological types of primary melanoma as well as metastatic melanoma and 3 types of benign nevi as representative examples of those subtypes most commonly found in patient populations contributed to providing this high level of performance.

The proteomic signatures established by HGMS may be utilized as a supplement to standard histology. Our successful use of FFPE tissue further supports the practicability of combining HGMS assay with histopathology in evaluation of melanocytic lesions. HGMS analysis could be particularly helpful in cases that are histologically equivocal, and a firm diagnosis of benign nevus or malignant melanoma cannot be made with absolute certainty. Work is ongoing in histologically ambiguous lesions, but based upon the results in this retrospective study, the proteomic signature appears applicable to a broad array of benign and malignant melanocytic lesions, including some that might prove challenging to classify by histopathology alone.

Implementations of the mass spectrometry system may be described within the context of a device configured to perform various steps, methods, and/or functionality in accordance with aspects of the present invention. It is to be appreciated that a mass spectrometry system including a computing device or computer system may be implemented by one or more computing devices. Implementations of the mass spectrometry system may be described in the context of a “device configured to”, wherein the term configured may be taken to mean that the device may implement computer-executable instructions that are executed to perform various steps, methods, and/or functionality in accordance with aspects of the present invention.

In general, a computer system or computing device may include one or more processors and storage devices (e.g., memory and disk drives) as well as various input devices, output devices, communication interfaces, and/or other types of devices. A computer system or computing device can also include a combination of hardware and software. It should be appreciated that various types of computer-readable storage media can be part of a computer system or computing device. As used herein, the terms “memory”, “computer-readable storage media” and “computer-readable storage medium” do not mean and unequivocally exclude a propagated signal, a modulated data signal, a carrier wave, or any other type of transitory computer-readable medium. The mass spectrometry system may include a processor configured to execute computer-executable instructions and a computer-readable storage medium (e.g., memory and/or additional hardware storage) storing computer-executable instructions configured to perform various steps, methods, and/or functionality in accordance with aspects of the present invention.

Computer-executable instructions may be embodied and/or implemented in various ways such as by a computer program (e.g., client program and/or server program), a software application (e.g., client application and/or server application), software code, application code, source code, executable files, executable components, routines, application programming interfaces (APIs), functions, methods, objects, properties, data structures, data types, and/or the like. Computer-executable instructions may be stored on one or more computer-readable storage media and can be executed by one or more processors, computing devices, and/or computer systems to perform particular tasks or implement particular data types in accordance with aspects of the present invention.

The mass spectrometry system may implement and utilize one or more program modules. Generally, program modules include routines, programs, objects, components, data structures, etc., that perform particular tasks or implement particular abstract data types.

The mass spectrometry system may be implemented as a distributed computing system or environment in which components are located on different computing devices that are connected to each other through network (e.g., wired and/or wireless) and/or other forms of direct and/or indirect connections. In such distributed computing systems or environments, tasks can be performed by one or more remote processing devices, or within a cloud of one or more devices, that are linked through one or more communications networks. In a distributed computing environment, program modules can be located in both local and remote computer storage media including media storage devices. Still further, the aforementioned instructions can be implemented, in part or in whole, as hardware logic circuits, which can include a processor.

The mass spectrometry system may be implemented by one or more computing devices such as computers, PCs, server computers configured to provide various types of services and/or data stores in accordance with aspects of the present invention. Exemplary sever computers can include, without limitation: web servers, front end servers, application servers, database servers, domain controllers, domain name servers, directory servers, and/or other suitable computers.

Components of the mass spectrometry system may be implemented by software, hardware, firmware or a combination thereof. For example, the mass spectrometry system may include components implemented by computer-executable instructions that are stored on one or more computer-readable storage media and that are executed to perform various steps, methods, and/or functionality in accordance with aspects of the present invention.

The mass spectrometry system may include a controller, memory, additional hardware storage, input devices, and output devices. Input devices may include one or more of the exemplary input devices described above and/or other type of input mechanism and/or device. Output devices may include one or more of the exemplary output devices described above and/or other type of output mechanism and/or device, such as a display.

The mass spectrometry system may contain one or more communication interfaces that allow the mass spectrometry system to communicate with other computing devices and/or other computer systems. The mass spectrometry system may include and/or run one or more computer programs implemented, for example, by software, firmware, hardware, logic, and/or circuitry of the mass spectrometry system. Computer programs can include an operating system implemented, for example, by one or more exemplary operating systems described above and/or other type of operating system suitable for running on computing device. Computer programs can include one or more applications.

The terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first set of one or more lines of code and may comprise a second “circuit” when executing a second set of one or more lines of code. As utilized herein, circuitry is “operable” to perform a function whenever the circuitry comprises the necessary hardware and code (if any is necessary) to perform the function, regardless of whether performance of the function is disabled, or not enabled (e.g., by a user-configurable setting, factory trim, etc.).

The terms “communicate” and “communicating” include both conveying data from a source to a destination and delivering data to a communications medium, system, channel, network, device, wire, cable, fiber, circuit, and/or link to be conveyed to a destination. The term “communication” as used herein means data so conveyed or delivered. The term “communications” as used herein includes one or more of a communications medium, system, channel, network, device, wire, cable, fiber, circuit, and/or link.

The terms “connect,” “connected,” and “connection” each mean a relationship between or among two or more devices, apparatuses, files, circuits, elements, functions, operations, processes, programs, media, components, networks, systems, subsystems, and/or means, constituting any one or more of: (i) a connection, whether direct or through one or more other devices, apparatuses, files, circuits, elements, functions, operations, processes, programs, media, components, networks, systems, subsystems, or means; (ii) a communications relationship, whether direct or through one or more other devices, apparatuses, files, circuits, elements, functions, operations, processes, programs, media, components, networks, systems, subsystems, or means; and/or (iii) a functional relationship in which the operation of any one or more devices, apparatuses, files, circuits, elements, functions, operations, processes, programs, media, components, networks, systems, subsystems, or means depends, in whole or in part, on the operation of any one or more others thereof.

The term “data” means any indicia, signals, marks, symbols, domains, symbol sets, representations, and any other physical form or forms representing information, whether permanent or temporary, whether visible, audible, acoustic, electric, magnetic, electromagnetic, or otherwise manifested. The term “data” is used to represent predetermined information in one physical form, encompassing any and all representations of corresponding information in a different physical form or forms.

The following aspects are disclosed in this application:

Aspect 1. A method for differentiating benign melanocytic nevi from malignant melanomas, the method comprising: (a) treating a skin lesion sample; (b) subjecting the skin lesion sample to mass spectrometry; (c) generating a mass spectrometry profile from the skin lesion sample; (d) comparing the mass spectrometry profile of the skin lesion sample to a reference profile, wherein the reference profile includes a statistical average profile from a plurality of known benign melanocytic nevi and/or a plurality of known malignant melanoma; and (e) classifying the skin lesion sample as a benign melanocytic nevus or a melanocytic malignant melanoma based on a level of similarity between the mass spectrometry profile of the skin lesion sample and the reference profile.

Aspect 2. The method of aspect 1 wherein the mass spectrometry is matrix-assisted laser desorption mass spectrometry.

Aspect 3. The method of aspect 1 or 2, wherein treating the skin lesion sample comprises preparing the skin lesion sample for mass spectrometry.

Aspect 4. The method of aspect 3, wherein the preparing the skin lesion sample comprises at least one of: (a) staining the skin lesion sample; (b) subjecting the skin lesion sample to deparaffinization and antigen retrieval; (c) subjecting the skin lesion sample to on-tissue tryptic digestion; and (d) applying a MALDI compatible matrix.

Aspect 5. The method according to any one of aspects 1 to 4, wherein treating the skin lesion sample comprises: (a) annotating a digital microscopy image of the reference profile, and; (b) imposing the annotated digital microscopy image of the reference profile upon the skin lesion sample prior to mass spectrometry.

Aspect 6. The method according to any one of aspects 1 to 5, wherein treating the skin lesion sample is performed before subjecting the skin lesion sample to mass spectrometry.

Aspect 7. The method according to any one of aspects 1 to 6, wherein subjecting the skin lesion sample to mass spectrometry and generating a mass spectrometry profile from the skin lesion samples are performed before treating the skin lesion sample.

Aspect 8. The method of aspect 7, wherein the treatment comprises: (a) staining the skin lesion sample; and (b) annotating a digital microscopy image of the skin lesion sample.

Aspect 9. The method of aspect 4, wherein the reference profile or sample is stained with hematoxylin and eosin.

Aspect 10. The method according to any one of aspects 1 to 9, comprising repeating the steps of (a)-(e) on the skin lesion sample.

Aspect 11. The method according to any one of aspects 1 to 10, wherein the skin lesion sample comprises melanocytic components.

Aspect 12. The method according to any one of aspects 1 to 11, wherein the skin lesion sample comprises stromal components, benign melanocytic nevi components, and/or malignant melanocytic components.

Aspect 13. The method according to any one of aspects 1 to 12, wherein both melanocytic and stromal components of the skin lesion sample are subjected to mass spectrometry.

Aspect 14. The method according to any one of aspects 1 to 13, comprising treating the patient with at least one of chemotherapy, immunotherapy, toxin therapy, surgery, and radiotherapy when the patient is identified as having malignant melanoma.

Aspect 15. The method according to any one of aspects 1 to 14, comprising performing histologic analysis on the skin lesion sample.

Aspect 16. The method according to any one of aspects 1 to 15, wherein the plurality of known benign melanocytic nevi and the plurality of known malignant melanoma are classified by immunohistochemical analysis, genetic analysis, patient outcome, or a combination thereof.

Aspect 17. The method according to any one of aspects 1 to 16, comprising immunohistochemical analysis on the skin lesion sample.

Aspect 18. The method according to any one of aspects 1 to 17, wherein the method has a sensitivity and a specificity of one of at least 75%, at least 80%, at least 90% and at least 95% in correctly classifying the skin lesion sample.

Aspect 19. A method for differentiating benign melanocytic nevi from malignant melanomas, the method comprising: (a) treating a skin lesion sample from a patient; (b) subjecting the skin lesion sample to mass spectrometry; (c) generating a mass spectrometry profile from the skin lesion sample; (d) comparing the mass spectrometry profile of the skin lesion sample to a reference profile, wherein the reference profile includes a statistical average profile from a plurality of known benign melanocytic nevi and/or a plurality of known malignant melanoma; and (e) classifying the skin lesion sample as a benign melanocytic nevus or a melanocytic malignant melanoma based on a level of similarity between the mass spectrometry profile of the skin lesion sample and the reference profile, wherein the statistical average profile is generated using a genetic algorithm which defines one or more markers.

Aspect 20. The method of aspect 19, the statistical average profile comprises markers represented by peptide peaks at one or more of m/z 683.47±0.2, m/z 738.45±0.2, m/z 960.57±0.2, m/z 983.61±0.2, m/z 1043.70±0.2, m/z 1110.64±0.2, m/z 1143. 63±0.2, m/z 1215.71±0.2, m/z 1254.73±0.2, m/z 1314.76±0.2, m/z 1454.86±0.2, m/z 1473.84±0.2, m/z 1489.94±0.2, m/z 1505.94±0.2, m/z 1507.90±0.2, m/z 1513.83±0.2, m/z 1519.97±0.2, m/z 1521.76±0.2, m/z 1569.89±0.2, m/z 1592.92±0.2, m/z 1633.91±0.2, m/z 1730.88±0.2, m/z 1878.02±0.2, and m/z 2187.17±0.2.

Molecular imaging and signature identification by mass spectrometry imaging may allow clinicians to look beyond classic histology. Gene expression may be useful for distinguishing melanocytic nevi from malignant melanomas. However, it does not always correlate with protein translation and does not account for post-translational modification. Since both protein expression level and post-translational modification state have fundamental effects on cellular function or dysfunction, it may be more meaningful to analyze proteins and peptides that are involved in the development and progression of a cancer. The parent proteins of the peptides that form the classification algorithm may be identified and serve as targets for treatment or for other applications such as immunohistochemical staining. A unique proteomic signature may be obtained for each disease that is studied by this approach. Mass spectrometry imaging (MSI) has the ability to discover molecular signatures of cancer and diseases that together result in robust diagnostic patterns.

In various embodiments, mass spectrometry imaging may be used to study benign melanocytic nevi and malignant melanomas in search of proteomic differences.

Further, proteomic signatures established using MSI classification may be used as a supplement to standard histology in evaluation of melanocytic lesions.

Mass spectrometry imaging and analysis may be useful in cases that are histologically equivocal, and a firm diagnosis of benign melanocytic nevus or malignant melanoma cannot be made with absolute certainty.

Mass spectrometry imaging may provide one or more of the following advantages: mass spectrometry works in formalin-fixed paraffin embedded tissue sections; mass spectrometry is a reliable method to differentiate benign nevi from malignant melanoma based on proteomic differences; mass spectrometry can help in the diagnosis of ambiguous cases by histology; and mass spectrometry is objective, fast, and relatively inexpensive.

As discussed above, 7 histological types of primary melanoma as well as metastatic melanoma and 3 types of benign nevi where evaluated to develop a classification algorithm using HGMS profiling and machine learning to provide proteomic molecular insight to aid in the diagnosis of challenging melanocytic lesions. The present invention showed that benign nevi and melanomas could be successfully distinguished using HGMS analysis based on detection of proteomic differences. HGMS was able to differentiate melanomas from nevi with 98.76% sensitivity and 99.65% specificity in the validation cohort with overall accuracy of 99.24%.

The proteomic signatures established by HGMS may be utilized as a supplement to standard histology. The use of FFPE tissue further supports the practicability of combining HGMS assay with histopathology in evaluation of melanocytic lesions. HGMS analysis may be particularly helpful in cases that are histologically equivocal, and a firm diagnosis of benign nevus or malignant melanoma cannot be made with absolute certainty. The proteomic signature may be applicable to a broad array of benign and malignant melanocytic lesions, including some that might prove challenging to classify by histopathology alone.

All documents cited herein are incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to the systems and methods described herein.

What is claimed is:

1. A method of performing mass spectrometry for differentiating benign melanocytic nevi from malignant melanomas, the method comprising:
(a) treating a skin lesion sample;
(b) subjecting the skin lesion sample to mass spectrometry;
(c) generating a mass spectrometry profile from the skin lesion sample;
(d) comparing the mass spectrometry profile of the skin lesion sample to a reference profile, wherein the reference profile includes a statistical average profile from at least one of a plurality of known benign melanocytic nevi and a plurality of known malignant melanoma; and
(e) classifying the skin lesion sample as a benign melanocytic nevus or a melanocytic malignant melanoma based on a level of similarity between the mass spectrometry profile of the skin lesion sample and the reference profile.

2. The method of claim 1, wherein the mass spectrometry is matrix-assisted laser desorption mass spectrometry.

3. The method of claim 1, wherein treating the skin lesion sample comprises preparing the skin lesion sample for mass spectrometry selected from:
(a) staining the skin lesion sample;
(b) subjecting the sample to deparaffinization and antigen retrieval prior to on-tissue tryptic digestion and matrix application using a robotic sprayer;
(c) subjecting the skin lesion sample to on-tissue tryptic digestion; and
(d) applying a MALDI compatible matrix.

4. The method of claim 1, wherein treating the skin lesion sample comprises:
(a) annotating a digital microscopy image of the reference profile, and;
(b) imposing the annotated digital microscopy image of the reference profile upon the skin lesion sample prior to mass spectrometry.

5. The method of claim 1, wherein treating the skin lesion sample is performed before subjecting the skin lesion sample to mass spectrometry.

6. The method of claim 1, wherein treating the sample is performed after subjecting the sample to mass spectrometry.

7. The method of claim 1, wherein subjecting the skin lesion sample to mass spectrometry and generating a mass spectrometry profile from the skin lesion samples are performed before treating the skin lesion sample.

8. The method of claim 7, wherein the treatment comprises:
(a) staining the skin lesion sample; and
(b) annotating a digital microscopy image of the skin lesion sample.

9. The method of claim 4, wherein the reference profile or sample is stained with hematoxylin and eosin.

10. The method of claim 1, comprising repeating the steps of (a)-(e) on the skin lesion sample.

11. The method of claim 1, wherein the skin lesion sample comprises melanocytic components.

12. The method of claim 1, wherein the skin lesion sample comprises at least one of stromal components, benign melanocytic nevi components, and malignant melanocytic components.

13. The method of claim 1, wherein both melanocytic and stromal components of the skin lesion sample are subjected to mass spectrometry.

14. The method of claim 1, comprising treating the patient with at least one of chemotherapy, immunotherapy, toxin therapy, surgery, and radiotherapy when the patient is identified as having malignant melanoma.

15. The method of claim 1, comprising performing histologic analysis on the skin lesion sample.

16. The method of claim 1, wherein the plurality of known benign melanocytic nevi and the plurality of known malignant melanoma are classified by immunohistochemical analysis, genetic analysis, patient outcome, or a combination thereof.

17. The method of claim 1, comprising immunohistochemical analysis on the skin lesion sample.

18. The method of claim 1, wherein the method has a sensitivity and a specificity of at least 75% in correctly classifying the skin lesion sample.

19. A method of performing mass spectrometry for differentiating benign melanocytic nevi from malignant melanomas, the method comprising:
(a) subjecting a skin lesion sample from a patient to on-tissue tryptic digestion;
(b) subjecting the skin lesion sample to mass spectrometry;
(c) generating a mass spectrometry profile from the skin lesion sample;
(d) comparing the mass spectrometry profile of the skin lesion sample to a reference profile, wherein the reference profile includes a statistical average profile from a plurality of known benign melanocytic nevi and a plurality of known malignant melanoma; and
(e) classifying the skin lesion sample as a benign melanocytic nevus or a melanocytic malignant melanoma based on a level of similarity between the mass spectrometry profile of the skin lesion sample and the reference profile,
wherein the statistical average profile is generated using a genetic algorithm which defines one or more markers.

20. The method of claim 19, the statistical average profile comprises markers represented by peptide peaks at one or more of m/z 683.47±0.2, m/z 738.45±0.2, m/z 960.57±0.2, m/z 983.61±0.2, m/z 1043.70±0.2, m/z 1110.64±0.2, m/z 1143. 63±0.2, m/z 1215.71±0.2, m/z 1254.73±0.2, m/z 1314.76±0.2, m/z 1454.86±0.2, m/z 1473.84±0.2, m/z 1489.94±0.2, m/z 1505.94±0.2, m/z 1507.90±0.2, m/z 1513.83±0.2, m/z 1519.97±0.2, m/z 1521.76±0.2, m/z 1569.89±0.2, m/z 1592.92±0.2, m/z 1633.91±0.2, m/z 1730.88±0.2, m/z 1878.02±0.2, and m/z 2187.17±0.2.
